(19) United States
(12) Patent Application Publication
(10) Pub. No.: US 2013/0090634 A1
(43) Pub. Date: Apr. 11, 2013

(54) CORNEAL INCISION USING A SURGICAL LASER

(71) Applicant: James Carlton Loden, Nashville, TN (US)

(72) Inventor: James Carlton Loden, Nashville, TN (US)

(21) Appl. No.: 13/267,485

(22) Filed: Oct. 6, 2011

(51) Int. Cl. A61B 18/20

(52) U.S. Cl. 606/4

(57) ABSTRACT

A lens cover for covering an applanation lens on a docking cone assembly to allow a conventional LASIK opthalmic laser surgical system perform a clear corneal incision for cataract surgery. The lens cover includes a slot that is shaped to correspond to the desired width of the clear corneal incision. Incident light from the opthalmic laser surgical system is partially allowed to pass through the slot, through the applanation lens, and into the patient's cornea.

### TECHNICAL FIELD

This application relates generally to eye surgery and more particularly to systems, devices and methods for forming a clear corneal incision.

### BRIEF DESCRIPTION OF THE DRAWINGS

The drawings, when considered in connection with the following description, are presented for the purpose of facilitating an understanding of the subject matter sought to be protected.

FIG. 1 illustrates an exploded perspective view of an embodiment of a docking cone assembly.

FIG. 2 illustrates a partially exploded view of the embodiment of the docking cone assembly of FIG. 1 with the applanation lens attached.

FIG. 3A illustrates a partial cross-sectional view of an embodiment of a lens cover positioned for installation onto an applanation lens.

FIG. 3B illustrates a partial cross-sectional view of an embodiment of a lens cover positioned on an applanation lens.

FIG. 4 illustrates a plan view of an embodiment of a lens cover.

FIG. 5 illustrates a perspective view of an embodiment of the lens cover of FIG. 4.

FIG. 6 illustrates a plan view of another embodiment of a lens cover.

FIG. 7 illustrates a perspective view of an embodiment of the lens cover of FIG. 6.

FIG. 8 illustrates a partial cross-sectional view of an embodiment of a lens cover.

FIG. 9 illustrates a detail partial cross-sectional view of the embodiment of a lens cover positioned on an applanation lens of FIG. 3B.

FIG. 10A illustrates a partial cross-sectional view of an embodiment of a docking cone assembly attached to an eye with a lens cover positioned for installation onto the applanation lens.

FIG. 10B illustrates a partial cross-sectional view of the embodiment of a docking cone assembly of FIG. 10A with the lens cover positioned on the applanation lens.

FIG. 11 illustrates a plan view of an alternative embodiment of a lens cover having a second slot defined therein.

FIG. 12 illustrates a plan view of an alternative embodiment of a lens cover having another embodiment of a second slot defined therein.

FIG. 13A illustrates a partial perspective view of an embodiment of a docking cone assembly with an embodiment of a lens cover positioned on the applanation lens.

FIG. 13B illustrates a partial perspective view of the embodiment of a docking cone assembly of FIG. 13A showing a tool engaging a tool cavity defined on the lens cover.

FIG. 13C illustrates a partial perspective view of the embodiment of a docking cone assembly of FIG. 13B showing a tool being used to rotate the lens cover into a desired position while the lens cover is positioned on the applanation lens.

FIG. 14 illustrates a partial cross-sectional view of an eye showing an embodiment of a clear corneal incision.

### DETAILED DESCRIPTION

While the present disclosure is described with reference to several illustrative embodiments described herein, it should be clear that the present disclosure should not be limited to such embodiments. Therefore, the description of the embodiments provided herein is illustrative of the present disclosure and should not limit the scope of the disclosure as claimed. In addition, while following description references corneal incision, it will be appreciated that the disclosure may be used with other types of laser surgery.

Cataract surgery generally removes a clouded or damaged lens from a patient's eye. A new lens may then be inserted into the eye to correct the patient's vision. All conventional types of cataract surgery require an incision in the cornea. In many applications, a clear corneal incision includes a small incision approximately in the plane of the cornea ranging from about 1.8 to about 3.5 mm in width. The incision passes completely through the cornea at an incision location near the limbus, forming a tunnel through which a surgeon may insert a tool for lens removal, such as a phacoemulsification tool. The tool may be used to break up and remove the opaque or clouded lens. Such clear corneal incisions for cataract surgery are conventionally formed using a small blade known as a keratome. The keratome, which is made of steel or diamond material, includes a blade width and shape substantially equal to the size and shape of the desired clear corneal incision. Traditionally, the keratome is manually inserted directly into the cornea by the surgeon to form corneal incision.

Conventional tools and free-hand methods for forming corneal incisions during cataract surgery using a keratome can cause abnormally large incisions, improper wound architecture leading to non-sealing leaky wounds, and descemet's detachments and trauma to the eye. Such trauma may result in improper postoperative sealing along the incision site.

Others have attempted to prevent such complications by providing keratomes and associated incision procedures that result in three-dimensional incisions. For example, some conventional keratomes and associated incision methods may produce a two-step or a three-step planed incision through the cornea. It is believed in the art that such three-dimensional incisions may reduce postoperative complications such as inadequate sealing or extended healing times. However, such conventional devices and methods still utilize a mechanical blade that involves mechanical contact to the cornea and surrounding tissues. Such mechanical contact makes further complications possible, including improper sealing, tearing and distortion. As a result, conventional clear corneal incisions may require suturing or extended recovery times.

Incisions for opthalmic surgery may also be performed in other types of surgeries using a laser instead of a mechanical keratome. For example, lasers are known in the art for use in laser-assisted in situ keratomileusis, or LASIK eye surgery, for correcting such refractive conditions as myopia, hyperopia or astigmatism. During a LASIK procedure, a corneal flap is created by forming a hinged, ring-shaped incision in the cornea. The corneal flap incision may be formed using a femtosecond laser rather than a conventional keratome. During flap creation, the laser is focused at a subsurface region of the cornea. The laser is pulsed at a predetermined frequency, and each series of pulses creates a small subsurface bubble in the cornea via intrastromal ablation (ISA). Multiple bubbles may be formed locally in a ring shape to provide a corneal flap that can be lifted by the surgeon for accessing the underlying corneal stroma. The corneal stroma may then be reshaped using an excimer laser. Following the surgery, the corneal flap may be closed.

LASIK eye surgery using a femtosecond laser system has recently become a common medical procedure. As such, femtosecond laser systems for LASIK eye surgery are commonly owned by eye surgeons. However, conventional ophthalmic surgery femtosecond laser systems for LASIK are generally configured for making a hinged, round flap-style incision for revealing the corneal stroma, and are not configured for producing a clear corneal incision of the type needed for cataract surgery. Because so many femtosecond laser systems for LASIK eye surgery are already in use, it would be beneficial if such devices were also configurable for performing both LASIK eye surgery, which requires a hinged, round flap incision, and cataract surgery, which requires a small clear corneal incision extending completely through the cornea.

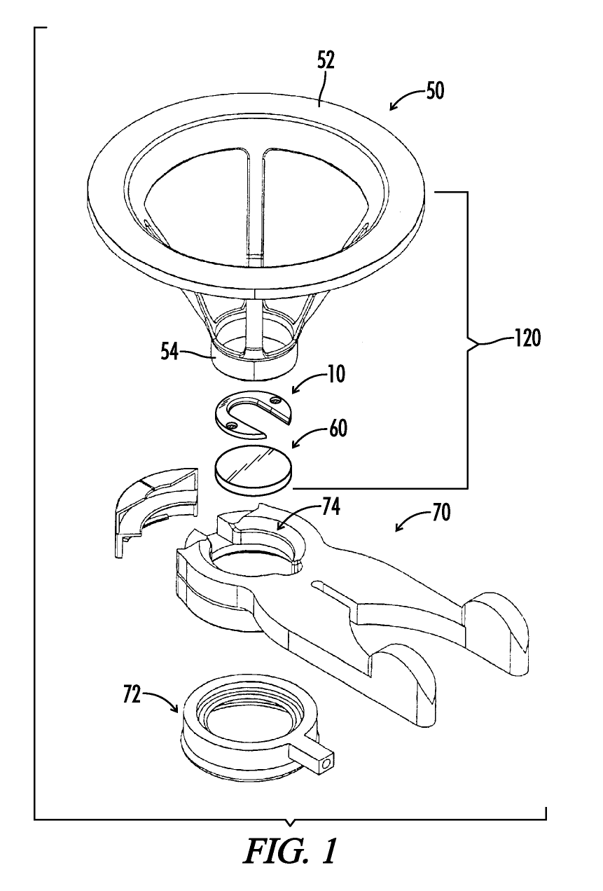

Referring now to the drawings, FIG. 1 illustrates an exploded perspective view of an embodiment of a docking cone assembly designated by the numeral 120. In addition, positional terms such as “upper,” “lower,” “side,” “top,” “bottom,” etc. refer to the apparatus when in the orientation shown in the drawing. The skilled artisan will recognize that the apparatus can assume different orientations when in use.

Referring further to FIG. 1, an exploded view of an embodiment of a docking cone assembly 120 is generally illustrated. Docking cone assembly 120 includes a docking cone 50, an applanation lens 60 and a lens cover 10. Docking cone assembly 120 is an ocular fixation device utilized for forming a mechanical interface between a patient's cornea and a surgical laser system. During eye surgery, an aperture of the laser surgical system from which laser light is emitted may be positioned above the docking cone assembly 120.

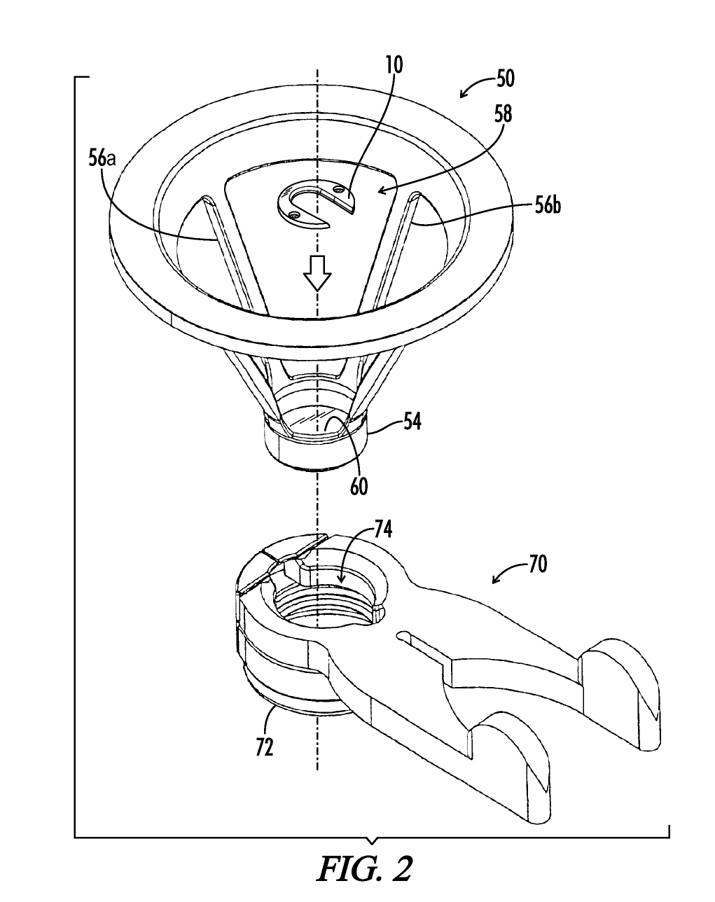

Docking cone 50 includes a tapered shape similar to an inverted frustrated cone in some embodiments. A base ring, or upper ring 52, may form an open, continuous circle in some embodiments and is configured for engagement with a surgical laser system. As seen in FIG. 2, a plurality of cone struts, or support struts 56a, 56b, etc. extend downwardly from the upper ring 52. A lower ring 54, or apex ring, is attached to the cone struts 56a, 56b, etc. Lower ring 54 also forms an open, continuous circle in some embodiments. Lower ring 54 has a lower ring diameter, and upper ring 52 has an upper ring diameter, wherein the lower ring diameter is generally less than the upper ring diameter in some embodiments. More specifically, lower ring 54 includes a substantially cylindrical shape in some embodiments and defines a wall thickness, thereby forming an inner lower ring diameter and an outer lower ring diameter. The cylindrical shape of the lower ring 54 in some embodiments provides a receptacle shaped for receiving an applanation lens 60.

Referring further to FIG. 1 and FIG. 2, applanation lens 60 includes a cylindrical lens that may be secured to the docking cone 50. Applanation lens 60 includes a substantially flat glass disk that may be used to provide a mechanical interface between an anterior corneal surface and a surgical laser system. Applanation lens 60 may have other shapes in other embodiments and applications. In some embodiments, applanation lens 60 is attached to lower ring 54 on docking cone 50. Lower ring 54 defines a lower ring inner diameter that corresponds to the outer diameter of applanation lens 60 in some embodiments. Applanation lens 60 may be held in place in lower ring 54 by a friction fit in some embodiments. In other embodiments, applanation lens 50 may be rigidly bonded to docking cone 50. In many embodiments, docking cone 50 and applanation lens 60 are provided for disposable use in a pre-assembled kit, or package.

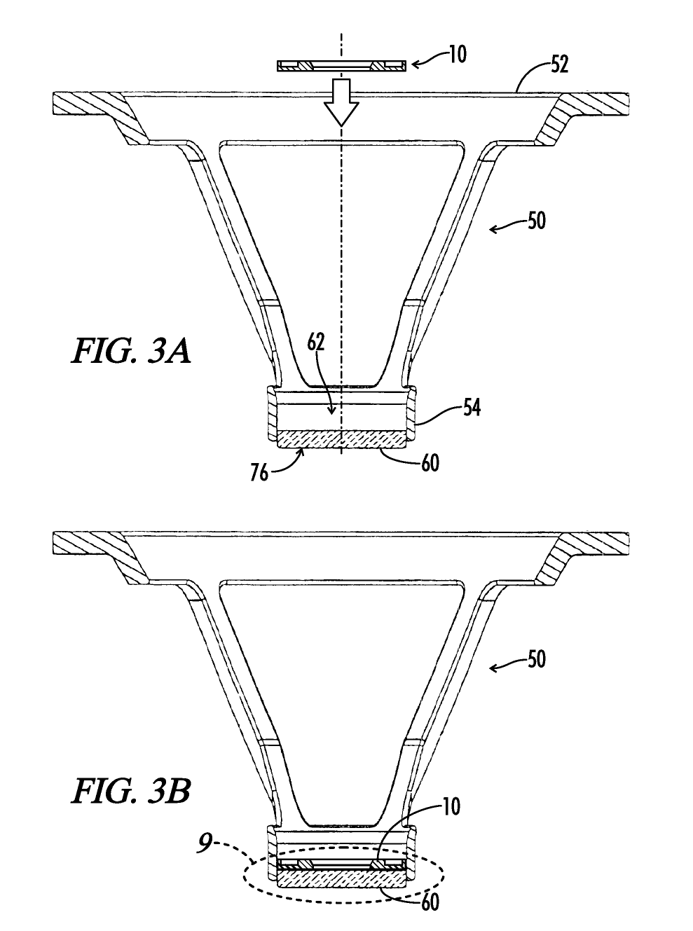

A lens cover 10 may be positioned on the applanation lens from above, as seen in FIGS. 1-3B. Lens cover 10 generally includes a circular disk having a bottom cover surface shaped to correspond to the upper lens surface of applanation lens 60 such that lens cover 10 engages in surface contact with applanation lens 60 while resting against applanation lens 60. In some embodiments, applanation lens 60 includes a substantially flat upper lens surface, and lens cover 10 includes a substantially flat bottom cover surface. As seen in FIG. 3B, and FIG. 9, in some embodiments, lens cover 10 rests flat against applanation lens 60. In some applications, docking cone 50 may already be engaged with a surgical laser system when lens cover 10 is positioned over applanation lens 60. Thus, lens cover 10 may be positioned over applanation lens 60 by inserting lens cover 10 through a strut opening 58 located between adjacent struts 56a, 56b on docking cone 50 and subsequently lowering lens cover 10 onto applanation lens 60. As seen in FIG. 3A, in other applications, lens cover 10 may be lowered onto applanation lens 60 through the annular opening in upper ring 52 on docking cone 50. Also seen in FIG. 3A, a lens cup 62 is defined between applanation lens 60 and lower ring 54. The lens cover 10 is generally dimensioned to be received in lens cup 62, as illustrated in FIG. 3B.

Referring further to FIG. 1 and FIG. 2, a gripper 70 may be positioned below docking cone assembly 120 in some embodiments. Gripper 70 provides a mechanical link between docking cone assembly 120 and a patient's eye. During laser cataract surgery, it is important that the operative target of the incident surgical laser beam does not move spatially relative to the beam source. Gripper 70 provides a fixture for maintaining the patient's eye at a stable location during surgery.

Gripper 70 includes a suction ring 72, or attachment ring, that may be secured to the patient's eye using a negative pressure applied through a pressure port on gripper 70. Suction ring 72 is attached to gripper 70. The suction ring 72 may be positioned directly against the patient's eye such that suction ring 72 surrounds the cornea and the cornea is exposed through a central opening in the suction ring 72 and may be accessed from above through gripper aperture 74. Docking cone assembly 120 may be lowered onto the gripper 70 such that the lower ring 54, together with applanation lens 60, advances into the gripper aperture 74. The gripper aperture 74 includes a gripper aperture inner diameter dimensioned to correspond to the outer diameter of lower ring 54. In some embodiments, gripper aperture inner diameter may be dimensioned slightly less than the outer diameter of lower ring 54 such that an interference fit is used to secure docking cone 50 to gripper 70. Gripper 70 may include first and second lever handles that may be selectively squeezed toward each other to temporarily widen the gripper aperture such that lower ring 54 may be received therein. Upon insertion of the lower ring 54 into the gripper aperture 74, first and second lever handles on gripper 70 are released, and docking cone 50 is secured to gripper 70.

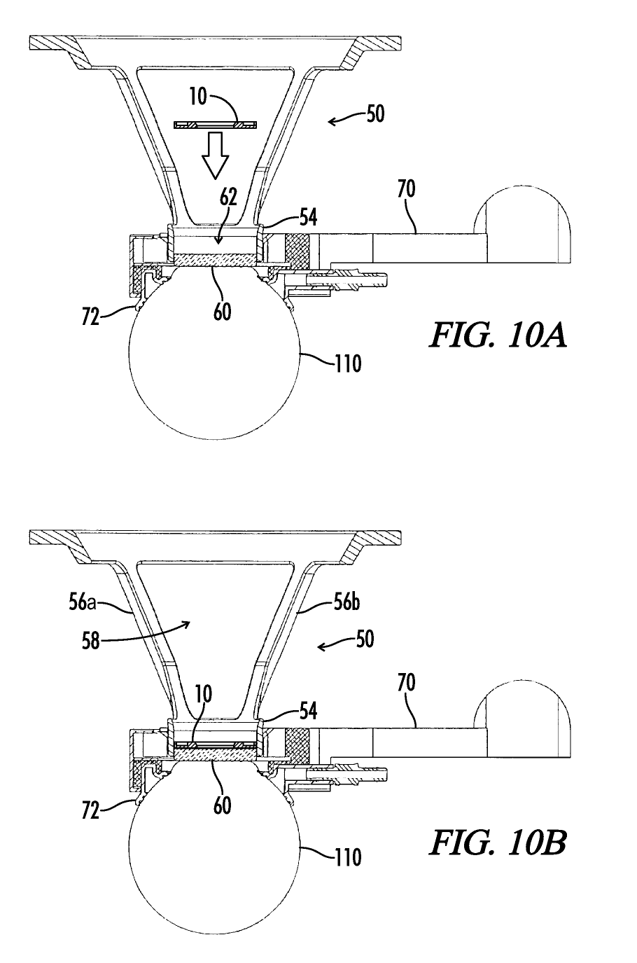

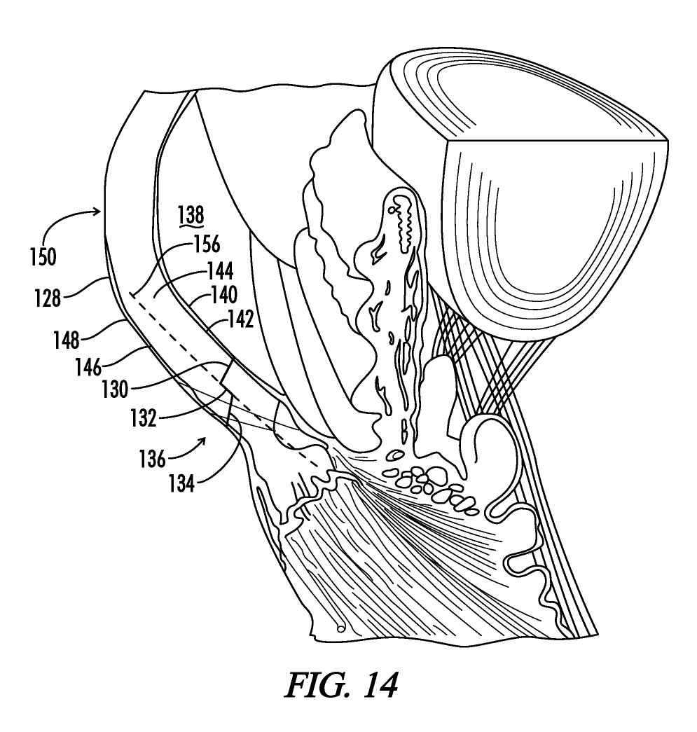

Referring to FIG. 10A, suction ring 72 may be positioned against a patient's eye 110 such that the patient's cornea extends slightly upward through a central opening in the suction ring 72 due to the curvature of the anterior surface of the cornea. When docking cone assembly 120 is positioned onto gripper 70, applanation lens 60 may slightly depress the cornea such that the shape of the anterior surface of the cornea corresponds to the shape of the applanation lens 60. In some applications, a substantially flat interface is formed between applanation lens 60 and the patient's cornea. More particularly, applanation lens 60 includes an applanation surface 76, labeled in FIG. 3A, facing away from lens cover 10. The applanation surface 76 is configured to directly contact the anterior surface of the patient's cornea when the docking cone assembly 120 is lowered onto the gripper 70, as seen in FIGS. 10A and 10B. In some applications, the applanation surface 76 is substantially flat for providing a uniform mechanical interface between applanation lens 60 and the anterior surface 150 of a patient's cornea, seen in FIG. 14.

In some applications, the docking cone 50, applanation lens 60 and gripper 70 with suction ring 72 may include commercially available parts such as the FS Disposable Patient Interface manufactured by AMO Manufacturing USA, LLC of Milpitas, Calif. and sold under the trade name IntraLase Femtosecond Technology and configured for use with commercially available IntraLase femtosecond surgical laser systems manufactured by Abbott Medical Optics of Santa Ana, Calif. and Milpitas, Calif. Although such conventional parts may be configured for LASIK eye surgery as supplied, combination of such parts with the devices and methods of the present disclosure, including lens cover 10, may render such parts suitable for performing cataract surgery.

The lens cover 10 provides a device and associated methods that allow a conventional opthalmic femtosecond surgical laser system to be used to make a clear corneal incision for cataract surgery. Conventionally, such femtosecond surgical laser systems have been used to form a circular, corneal flap for LASIK eye surgery. However, by partially blocking some of the incident laser light, or incident laser beam, emitted from the femtosecond surgical laser system using lens cover 10, such conventional systems may be used in additional applications requiring a smaller incision that extends completely through the cornea, such as a clear corneal incision for insertion of a phacoemulsification device for removing a cataracted lens.

In some embodiments, lens cover 10 comprises an opaque and a nonreflective material such as polyoxymethylene, or Delrin. Lens cover 10 may include any other suitable opaque and nonreflective material known in the art, including various types of woods, plastics, metals.

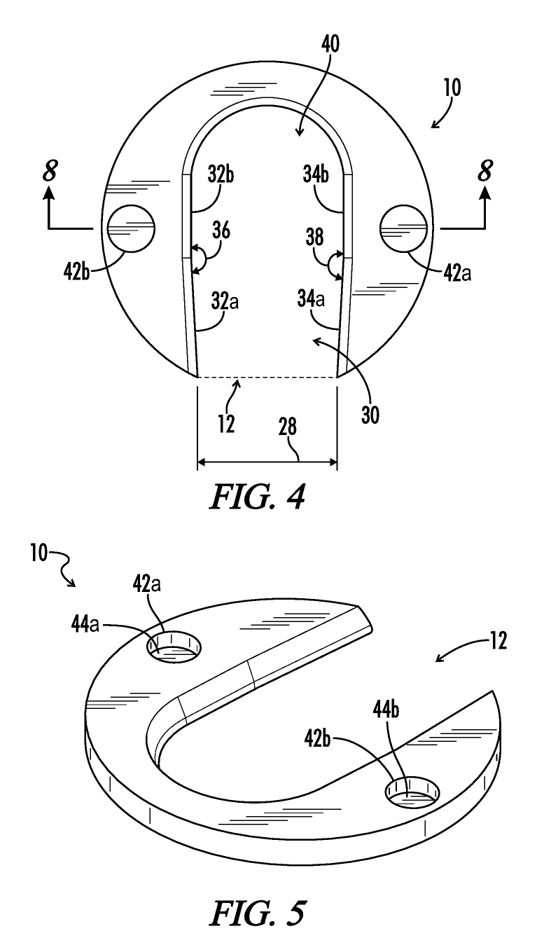

Referring to FIGS. 4-9, Lens cover 10 generally includes a slot 12 defined in the lens cover 10. Slot 12 includes a slot width 28. In some embodiments, slot 12 is open to a circumferential edge of lens cover 10, forming an opening along the perimeter of lens cover 10. In other embodiments, slot 12 may not extend entirely to the circumferential edge of lens cover 10, and may include a clearance hole defined in lens cover 10. Slot 12 may be described as a radial slot 12 in some embodiments. Slot 12 may be characterized as a radial slot by extending from an interior location on lens cover 10 to a location on or near the periphery of lens cover 10 such that the first end of the slot is located at a radial position closer to the center of lens cover 10 than the second end of the slot. Slot 12 need not extend exactly along a radius of lens cover 10 to be considered a radial slot.

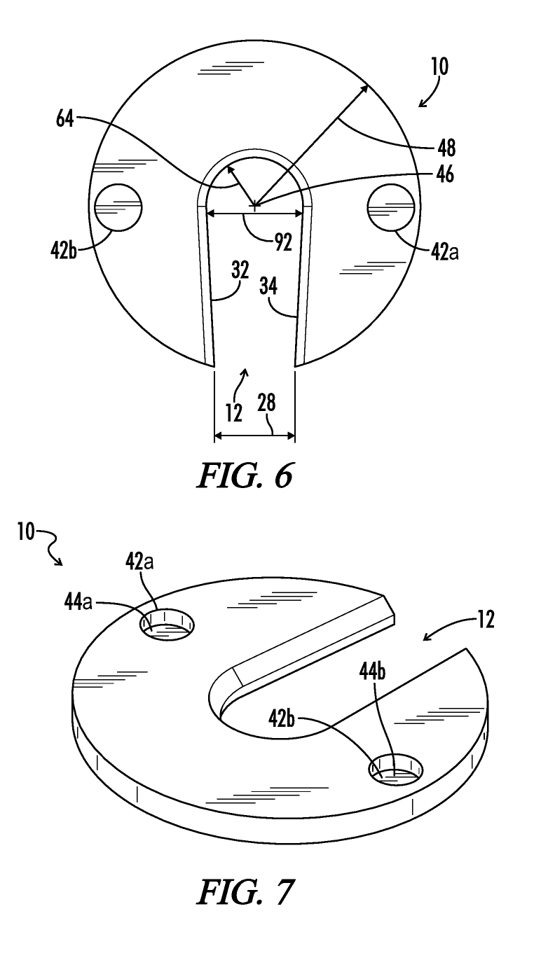

A slot width 28 is defined in lens cover 10. In some embodiments, slot width 28 may be substantially uniform. A substantially uniform slot width 28 forms substantially parallel slot edges, as seen for example in FIG. 12. In other embodiments, slot width 28 may taper in a narrowing fashion as slot 12 progresses toward the outer perimeter of lens cover 10, as seen for example in FIG. 6. In alternative embodiments, not shown, slot 12 may taper in an expanding fashion as slot 12 progresses toward the outer perimeter of lens cover 10. Slot 12 generally includes a first slot edge 32 and a second slot edge 34. First and second slot edges generally face each other across slot 12. In some embodiments, slot 12 may include a first slot width 28 and a second slot width 92, wherein second slot width 92 is located closer to the center of lens cover 10 than first slot width 28. In some embodiments, second slot width 92 is larger than first slot width 28. Slot 12 also includes an interior curved region in some embodiments positioned between first and second slot edges 32, 34. The interior curved region of slot 12 includes a semicircular shape in some embodiments having a substantially constant interior curved region radius 64. In some embodiments the ratio of slot width 28 to interior curved region radius 64 is between about 1.9 to about 2.1.

Referring now to FIG. 4, in some additional embodiments, slot 12 may include a more complex profile including an inner slot region 40 and an outer slot region 30. Inner slot region 40 includes a first inner slot edge 32b and a second inner slot edge 34b substantially facing each other across slot 12. Outer slot region 30 includes a first outer slot edge 32a and a second outer slot edge 34a substantially facing each other across slot 12 at a location closer to the perimeter of lens cover 10 than first and second inner slot edges 32b, 34b. In some embodiments, first inner slot edge 32b is oriented at a first slot edge angle 36 relative to first outer slot edge 32a. In some embodiments, first slot edge angle 36 is less than 180 degrees. In additional embodiments, first slot edge angle 36 is between about 175 degrees and about 180 degrees. Similarly, in some embodiments, second inner slot edge 34b is oriented at a second slot edge angle 38 relative to second outer slot edge 34a. In some embodiments, second slot edge angle 38 is less than 180 degrees. In additional embodiments, second slot edge angle 38 is between about 175 degrees and about 180 degrees.

Lens cover 10 rests against applanation lens 60, and slot 12 is shaped to partially allow incident light from a surgical laser to pass through lens cover 10. For example, as seen in FIG. 9, incident laser light, or an incident surgical laser beam 100, from an opthalmic surgical laser system is directed downward toward applanation lens 60. Incident surgical laser beam 100 from the surgical laser system may include a beam width greater than the slot width 28. Because lens cover 10 includes a generally opaque material in some embodiments, a portion of incident surgical laser beam 100 is partially blocked by lens cover 10, and another portion of the incident surgical laser beam 100 is allowed to pass through slot 12 on lens cover 10. As such, lens cover 10 includes a slot 12 that is shaped to partially allow light from the surgical laser to pass through the lens cover 10. The transmitted portion 104 of incident surgical laser beam 100 may be focused at a location on or within the corneal tissue for forming a clear corneal incision in the patient's eye. The blocked portion 102 of incident surgical laser beam 100 does not pass through applanation lens 60. Slot width 28 is dimensioned to correspond to the desired size of the clear corneal incision. For example, if the desired clear corneal incision width is desired to be 2.5 mm, then slot width 28 is dimensioned to be about 2.5 mm. In some embodiments, slot width 28 may be between about 1.0 mm to about 4.0 mm. In other embodiments, slot width 28 may be between about 2.0 mm to about 3.0 mm. In further embodiments, slot width 28 is between about 2.2 mm to about 2.7 mm. In various other embodiments, slot width 28 is dimensioned to correspond to a clear corneal incision size and may be 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, 3.2 mm, 3.3 mm, 3.4 mm, 3.5 mm, 3.6 mm, 3.7 mm, 3.8 mm, 3.9 mm, 4.0 mm, or greater depending on the desired width of the clear corneal incision.

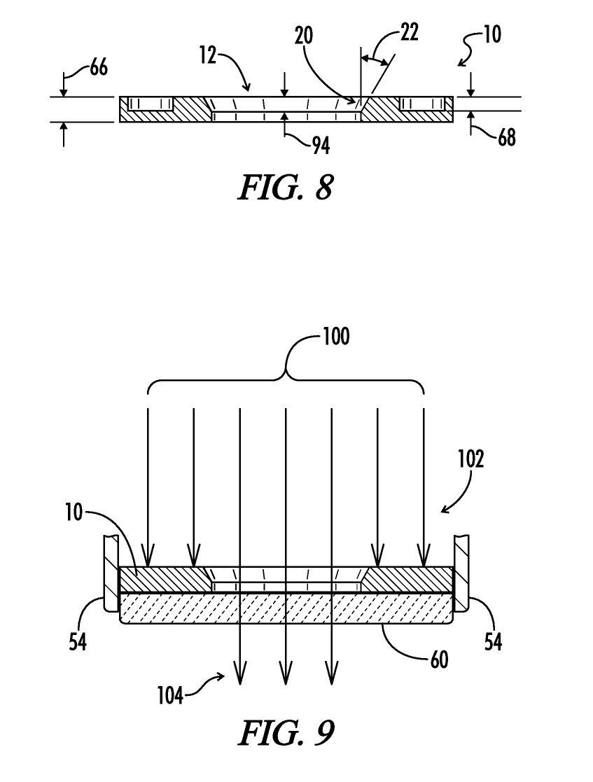

In some applications, it has been observed that first and second slot edges 32, 34 having a vertical profile may interfere with the transmission of incident laser light through slot 12, causing undesirable edge effects that may decrease the quality of the resulting clear corneal incision. An incident laser beam from a surgical laser system, such as an opthalmic femtosecond laser, may include multiple beam segments oriented at various angles to provide focused light at a desired intra-corneal spatial location in the corneal tissue where the multiple beam segments intersect. Vertical slot edges in some embodiments do not provide sufficient clearance for angled segments of the incident laser to pass through to the corneal tissue, causing undesired edge effects. To overcome such undesired edge effects associated with vertical slot edge profiles in some applications, lens cover 10 may include a slot 12 having one or more slot edges 32, 34 positioned adjacent slot 12, wherein at least one slot edge includes a non-perpendicular beveled region 20, seen in FIG. 8. Beveled region 20 generally defines a bevel angle 22 relative to a vertical reference axis oriented substantially perpendicular to the plane of the top surface of lens cover 10. In some embodiments, beveled region 20 does not extend across the entire height of the slot edge. Instead, beveled region 20 in some embodiments includes a beveled region height 94 less than the lens cover thickness 66. Lens cover thickness 66 in some embodiments is between about 0.5 mm and about 2.5 mm. In other embodiments, lens cover thickness 66 is between about 0.6 mm and about 0.8 mm. In a further embodiment, lens cover thickness 66 is about 0.76 mm. In various other embodiments (not shown), beveled region 20 includes a beveled region height 94 substantially equal to the lens cover thickness 66. In further embodiments, the ratio of the lens cover thickness 66 to the beveled region height 94 is between about 1.1 and about ten. In other embodiments, the ratio of the lens cover thickness 66 to the beveled region height 94 is between about 1.4 and about 1.6. In various other embodiments not shown, beveled region 20 may include a convex or concave curved profile not shown.

Referring further to FIG. 8, in some embodiments, bevel angle 22 is between about ten degrees and about eighty degrees. In further embodiments, bevel angle 22 is between about fifteen degrees and about forty-five degrees. In additional embodiments, a bevel angle 22 of about thirty degrees sufficiently allows passage of focused incident laser light to adequately form a clear corneal incision.

Referring further to FIGS. 10A and 10B, lens cover 10 may be positioned over and lowered onto applanation lens 60 after the suction ring 72 has been installed on the patient's eye 110 and after docking cone assembly 120 has been secured to gripper 70. In such applications, lens cover 10 may be inserted through a strut opening 58 between adjacent struts 56a, 56b and subsequently lowered onto applanation lens. In other embodiments, lens cover 10 may be positioned on applanation lens 60 before docking cone assembly 120 is secured to gripper 70.

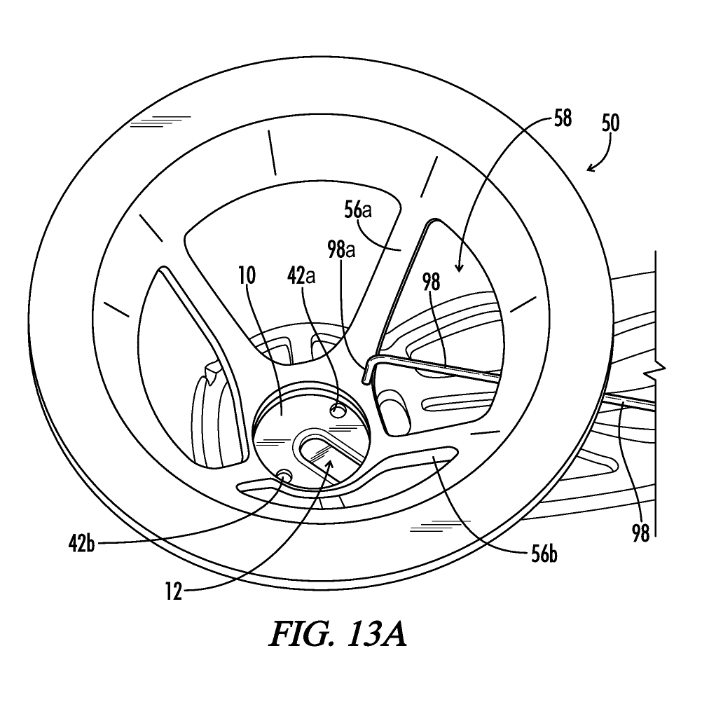

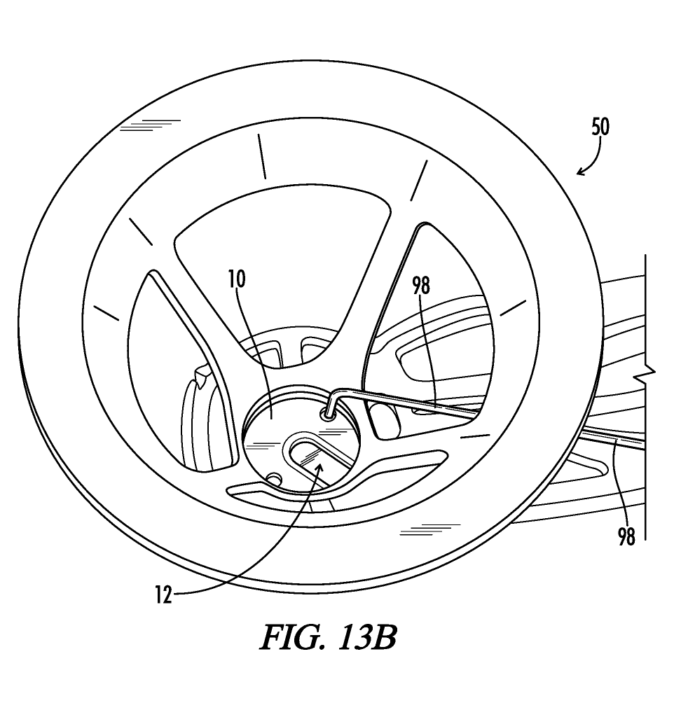

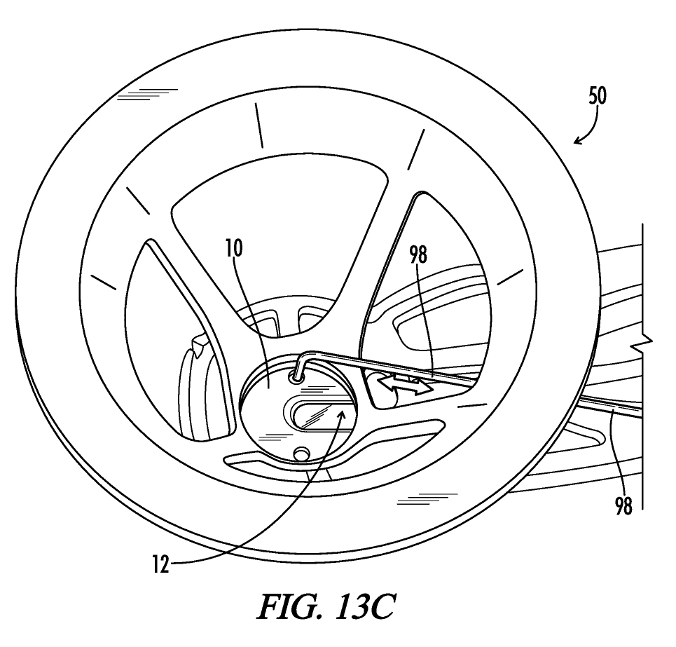

After lens cover 10 is installed onto applanation lens 60, slot 12 may not be angularly aligned with the desired location of the clear corneal incision. As such, it is generally desirable for a user to be able to angularly/rotationally reposition lens cover 10 relative to docking cone 50 and applanation lens 60 prior to irradiation by the surgical laser system. In some applications, a tool 98 may be inserted between adjacent struts 56a, 56b on docking cone 50 to angularly rotate lens cover 10 as it rests against applanation lens 60, as seen in FIG. 13A. In some applications, a user may engage distal tool tip 98a with any structure on lens cover 10 to rotate lens cover 10. For example, a user may engage distal tool tip 98a with a slot edge on slot 12 for rotating lens cover 10. However, because slot 12 is a clearance slot, there is potential for distal tool tip 98a to contact applanation lens 60, which could scratch or otherwise damage applanation lens 60. Thus, in some embodiments, the present disclosure provides a lens cover 10 having one or more tool recesses 42a, 42b defined therein.

Each tool recess 42a, 42b includes a tool recess depth 68, seen in FIG. 8. Each tool recess depth is less than lens cover thickness 66 in some embodiments. As such, a first tool recess floor 44a is formed at the bottom of first tool recess 42a, and a second tool recess floor 44b is formed at the bottom of second tool recess 42b in some embodiments. In such embodiments, each tool recess may be described as a blind axial hole open to the surface of the lens cover 10 facing away from applanation lens 60. Each first and second tool recess floor 44a, 44b may be integral to lens cover 10 and may prevent distal tool tip 98a from contacting applanation lens 60 when distal tool tip 98a is inserted into first or second tool recess 42a, 42b. Additionally, each tool recess floor 44a, 44b prevents incident laser beam 100 from passing through lens cover 10 in the vicinity of each tool recess 42a, 42b. As seen in FIG. 13B, distal tool tip may be manually inserted into a tool recess. Following engagement of distal tool tip 98a with a tool recess, tool 98 may be moved forward or backward to adjust the angular position of lens cover 10 such that slot 12 becomes aligned with the desired site of the clear corneal incision. Lens cover 10 may be dimensioned such that it is able to freely spin inside the lower ring 54 on docking cone 50 when engaged by tool 98. In some embodiments, lens cover 10 includes a lens cover diameter of about 11.0 mm. In other embodiments, lens cover 10 includes a lens cover diameter between about 9.0 mm and about 13.0 mm.

During cataract surgery, it may be desirable to form a second incision, or a second clear corneal incision, in the patient's eye. The second incision may be used for a paracentesis port for such applications as regulating the volume and pressure of fluid in the anterior chamber of the eye during removal of the cataracted lens. In other embodiments, the second incision may be used to insert a second tool such as a chopper for fragmenting the damaged lens for enhanced phacoemulsification. In some applications, it may be desirable to create the second incision in the cornea using the same femtosecond laser that is used to create the clear corneal incision. By using a laser to create the second incision, mechanical trauma to the eye associated with formation of the incision may be reduced.

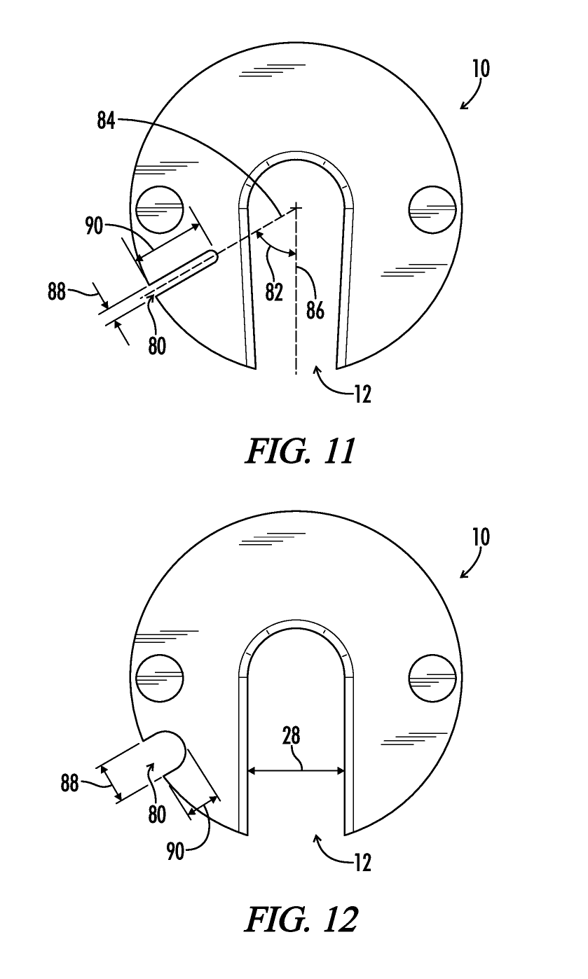

Referring now to FIG. 11 and FIG. 12, in some embodiments, lens cover 10 includes a side opening 80 shaped for partially allowing a portion of the incident laser beam to pass through lens cover 10 at a location different than the location of slot 12. Side opening 80 allows passage of incident laser light for forming the second clear corneal incision, or paracentesis port. In many applications, the second clear corneal incision, which is associated with side opening 80, may be smaller than the first clear corneal incision, which is associated with slot 12. As such, side opening 80 includes an opening width 88 and an opening length 90. In some embodiments, opening width 88 is between about 0.25 mm to about 2.0 mm. In other embodiments, opening width is about 1.0 mm. Opening length 90 in some embodiments may range between about 1.0 mm and about 3.0 mm. Also seen in FIG. 11, in some embodiments, side opening 80 is a radial opening that is offset from slot 12 by an opening offset angle 82. Opening offset angle 82 is measured as the angle between an opening reference axis 84 extending along a midpoint between opposing edges of side opening 80 and a slot reference axis 86 extending along a midpoint between opposing slot edges on slot 12. In some embodiments, opening offset angle 82 is between about thirty and about ninety degrees. In other embodiments, opening offset angle 82 is about sixty degrees.

Referring now to FIG. 14, in some embodiments, the devices and methods of the present disclosure may be used with a surgical laser system to produce a three-step planed clear corneal incision 136 for cataract surgery. A three-step planed clear corneal incision involves making a clearance incision, or tunnel, completely through the various layers of the corneal such that a tool may be inserted therethrough. A three-step planed corneal incision typically includes a complex, three-dimensional profile that decreases postoperative complications such as leakage and may lead to faster healing times. Conventionally, a mechanical keratome may be used to perform a three-step planed clear corneal incision. However, the present disclosure provides devices and methods for forming such incisions using a surgical laser, such as a femtosecond laser.

Referring further to FIG. 14, a three-step planed clear corneal incision 136 begins by making a first step incision 130 at the anterior chamber 138 cutting through the endothelium 140 and Descement's membrane 142 of the cornea. The first step incision 130 continues into the corneal stroma 144. At a midpoint through the cornea at a location in the corneal stroma 144, the clear corneal incision changes orientation, forming a second step 132 aligned substantially parallel to a local corneal plane 156 extending halfway through the cornea. The second step 132 may be described as a horizontal lamellar incision. The second step 132 is not truly horizontal, but is substantially parallel to the local orientation of the cornea at the incision site. The incision extends anteriorly from the second step away from the eye through the anterior stroma, through the Bowman's membrane 146 and through the epithelium 148. Each step of the incision may be formed by a focused femtosecond laser such as an IntraLase femtosecond laser.

In further embodiments, the present disclosure provides a method of forming a clear corneal incision in a patient's eye. The method includes the steps of: (a) providing a docking cone assembly having an applanation lens over a patient's cornea such that the applanation lens contacts the cornea; (b) positioning a lens cover having a slot defined therein on the applanation lens on the side of the applanation lens opposite the cornea; (c) irradiating the lens cover and applanation lens with a laser light beam from an opthalmic surgical laser system such that a first part of the incident laser light beam is blocked by the lens cover and a second part of the laser light beam passes through the slot; and (d) forming a clear corneal incision through the cornea. In some embodiments, the clear corneal incision is a three-step planed incision. In further embodiments, the method includes rotating the lens cover relative to the applanation lens such that the slot is positioned over, or angularly aligned with, the desired location of the clear corneal incision. In additional embodiments, the laser light beam includes pulsed light from a femtosecond laser.

In further embodiments, the present disclosure provides a method of retrofitting a LASIK femtosecond laser ophthalmic surgery system configured for flap creation to perform a clear corneal incision for cataract surgery. The method includes the steps of: (a) providing a lens cover having a slot defined therein; (b) providing a LASIK femtosecond laser ophthalmic surgery system configured for flap creation, the system including a laser source and an applanation lens; (c) positioning the lens cover between the laser source and the applanation lens; and (d) adjusting the position of the lens cover to correspond to the desired location of the clear corneal incision.

In some further embodiments, the present disclosure provides a method of modifying a docking cone assembly to block a portion of an incident laser beam for forming a clear corneal incision in a patient's eye. The method includes the steps of: (a) providing a docking cone assembly having a docking cone with an upper ring and a lower ring, the upper ring having a larger diameter than the lower ring and being positioned between the lower ring and a femtosecond surgical laser system, the lower ring including cylindrical shape and including an applanation lens secured transversely therein, the applanation lens and the lower ring forming a lens cup; and (b) positioning a lens cover in the lens cup such that the lens cover rests against the applanation lens, the lens cover defining a radial slot, wherein the radial slot is dimensioned to correspond to the width of the clear corneal incision. In some embodiments the upper ring may be attached or in contact with the femtosecond surgical laser system.

1. A lens cover apparatus for selectively blocking laser light from a surgical laser, which forms a corneal incision in a patient's eye, the apparatus comprising: an opaque lens cover, wherein the opaque lens cover forms at least one slot that is shaped to partially allow light from the surgical laser to pass through the lens cover, and wherein the at least one slot is shaped and dimensioned to correspond to a desired corneal incision.

2. The apparatus of claim 1, further comprising: a slot edge formed on the lens cover adjacent the slot, wherein the slot edge includes a non-perpendicular beveled region defining a bevel angle.

3. The apparatus of claim 2, wherein the bevel angle is between about ten degrees and about sixty degrees.

4. The apparatus of claim 2, wherein the bevel angle is about thirty degrees.

5. The apparatus of claim 1, further comprising: the slot including an outer slot region having a first slot width and an inner slot region having a second slot width.

6. The apparatus of claim 5, wherein: the first slot region includes a substantially uniform first slot width profile and the second slot region includes a tapered second slot width profile.

7. The apparatus of claim 1, further comprising: a tool recess defined in the lens cover.

8. The apparatus of claim 7, wherein the tool recess defines a blind axial hole in the lens cover.

9. The apparatus of claim 1, wherein the lens cover includes a thickness between about 0.5 mm and about 1.5 mm.

10. The apparatus of claim 1, wherein the lens cover comprises polyoxymethylene.

11. The apparatus of claim 1, wherein the slot width is between about 1.0 mm to about 4.0 mm.

12. The apparatus of claim 11, wherein the slot width is between about 2.0 mm to about 3.0 mm.

13. The apparatus of claim 11, wherein the slot width is between about 2.2 mm to about 2.7 mm.

14. The apparatus of claim 1, further comprising: an applanation lens positioned adjacent the lens cover, the applanation lens including an applanation surface facing away from the lens cover, the applanation surface configured to contact the anterior surface of the patient's cornea.

15. A docking cone assembly adapted to couple a patient's eye to a surgical laser system for forming a corneal incision, the docking cone assembly comprising:
a docking cone having an upper ring and a lower ring, wherein the upper ring is placed between the lower ring and the surgical laser system and the lower ring is placed between the upper ring and the patient's eye;
an applanation lens disposed in the lower ring, the applanation lens including an applanation surface configured to contact the anterior surface of the patient's cornea; and
an opaque lens cover disposed between the upper ring and the applanation lens, wherein the opaque lens cover is further configured to form at least one slot to allow a limited amount of laser light to pass through the applanation lens and to correspond to a desired corneal incision.

16. The apparatus of claim 15, wherein the slot width is between about 1.5 mm and about 3.5 mm.

17. The apparatus of claim 15, wherein the lens cover rests against the applanation lens.

18. The apparatus of claim 15, wherein the lens cover is angularly moveable relative to the applanation lens.

19. The apparatus of claim 15, further comprising:
a tool recess defined in the lens cover, the tool recess defining a blind hole in the lens cover.

20. A method of forming a clear corneal incision in a cornea, comprising:
(a) providing a docking cone assembly having an applanation lens over a patient's cornea such that the applanation lens contacts the cornea;
(b) positioning a lens cover having at least one slot defined therein on the applanation lens on the side of the applanation lens opposite the cornea;
(c) irradiating the lens cover and applanation lens with a laser light beam from a surgical laser system such that a first part of the laser light beam is blocked by the lens cover and a second part of the laser light beam passes through the at least one slot; and
(d) forming at least one clear corneal incision in the cornea.

21. The method of claim 20, wherein the clear corneal incision is a three-step planed clear corneal incision.

22. The method of claim 20, further comprising:
rotating the lens cover relative to the applanation lens such that the slot is positioned over the desired location of the clear corneal incision.

23. A method of retrofitting a LASIK femtosecond laser ophthalmic surgery system configured for flap creation to perform a clear corneal incision for cataract surgery, comprising:
(a) providing a lens cover having a slot defined therein;
(b) providing a LASIK femtosecond laser ophthalmic surgery system configured for flap creation, the system including a laser source and an applanation lens;
(c) positioning the lens cover between the laser source and the applanation lens; and
(d) adjusting the position of the lens cover to correspond to the desired location of the clear corneal incision.

24. The method of claim 23, wherein the lens cover comprises an opaque material.

25. The method of claim 23, wherein the lens cover rests on the applanation lens.

26. The method of claim 23, wherein the slot includes a beveled edge.

27. The method of claim 23, further comprising:
irradiating the lens cover with light from the laser opthalmic surgery system.

28. The method of claim 23, wherein the LASIK femtosecond laser ophthalmic surgery system includes an IntraLase FS Laser System.

29. The method of claim 23, wherein the clear corneal incision is between about 1.0 mm and about 3.5 mm.

30. The method of claim 23, wherein the clear corneal incision includes a three-step planed clear corneal incision.

31. The method of claim 23, further comprising:
providing a tool recess defined in the lens cover, wherein the tool recess includes a concave shape open to the side of the lens cover facing away from the applanation lens;
inserting a tool into the tool recess; and
rotating the lens cover to a desired angular orientation using the tool.

32. A method of modifying a docking cone assembly to block a portion of an incident laser beam for forming a clear corneal incision in a patient's eye, comprising:
(a) providing a docking cone assembly having a docking cone with an upper ring and a lower ring, the upper ring having a larger diameter than the lower ring and being positioned between the lower ring and a femtosecond surgical laser system, the lower ring including cylindrical shape and including an applanation lens secured transversely therein, the applanation lens and the lower ring forming a lens cup; and
(b) positioning a lens cover in the lens cup such that the lens cover rests against the applanation lens, the lens cover defining a radial slot, wherein the radial slot is shaped and dimensioned to correspond to desired specifics of the clear corneal incision.

33. The method of claim 32, wherein the docking cone includes a plurality of docking cone struts extending from the lower ring to the upper ring.

34. The method of claim 32, further comprising:
rotating the lens cover such that the angular position of the slot corresponds to the desired angular location of the clear corneal incision.
